(19) United States
(12) Patent Application Publication
(10) Pub. No.: US 2013/0053872 A1
(43) Pub. Date: Feb. 28, 2013

(54) DEVICE AND METHOD FOR PREVENTING BLOOD FLOW INTO AN ANEURYSM

(71) Applicant: Palle Munk HANSEN, Bjaeverskov (DK)

(72) Inventor: Palle Munk HANSEN, Bjaeverskov (DK)

(73) Assignees: COOK MEDICAL TECHNOLOGIES LLC, Bloomington, IN (US); WILLIAM COOK EUROPE, Bjaeverskov (DK)

(21) Appl. No.: 13/215,802

(22) Filed: Aug. 23, 2011

(51) Int. Cl. A61B 17/00

(52) U.S. Cl. 606/151

(57) ABSTRACT

The present invention provides an occlusion device for preventing blood flow from a blood vessel into an aneurysm through an opening in a blood vessel wall. The occlusion device includes an occluding sheet having a rolled configuration and a framework disposed with the occluding sheet and biased to unroll the occluding sheet. The present invention also provides a method of preventing blood flow from a blood vessel into an aneurysm using the occlusion device of the present invention.

### BACKGROUND

The present invention relates to medical devices. More particularly, the present invention relates to an occlusion device for preventing blood flow into an aneurysm and a method for preventing blood flow into an aneurysm using the occlusion device of the present invention.

Cerebral aneurysms are weak, bulging spots in an artery of the brain. If left untreated, cerebral aneurysms can enlarge and rupture. Treatment of a cerebral aneurysm is generally intended to reduce the pressure on the walls of the aneurysm to reduce the risk that the aneurysm will rupture. Most commonly, such treatment involves the placement of an embolization coil in the aneurysm. Embolization coils generally reduce the risk of aneurysm enlargement and rupture. In some cases, however, aneurysm enlargement continues when blood flows into the aneurysm and exerts pressure on the embolization coil.

### SUMMARY

The present invention generally provides an occlusion device for preventing blood flow from a blood vessel into an aneurysm. The invention also provides a method of preventing blood flow from a blood vessel into an aneurysm using the occlusion device of the present invention. Embodiments of the present invention enable interventionalists to prevent aneurysm expansion and rupture in patients by relieving pressure on the walls of the aneurysm.

In one embodiment, the present invention provides a method of preventing blood flow from a blood vessel into an aneurysm through an opening in a blood vessel wall. The method involves disposing a delivery system in a patient's vasculature. The delivery system includes an outer catheter having a proximal end, a distal end, and a delivery lumen formed therethrough. The distal end of the outer catheter is disposed in the blood vessel adjacent to the opening. The method further involves deploying an occlusion device from the delivery lumen into the blood vessel through the distal end of the outer catheter. The occlusion device includes an occluding sheet and a framework disposed with the occluding sheet. The occluding sheet has a first dimension and a second dimension. The occluding sheet has a rolled configuration and an unrolled configuration. The occluding sheet is rolled along the first dimension to define the rolled configuration. The occluding sheet has a first surface and a second surface. The first surface faces outwardly when the occluding sheet is in the rolled configuration. The second surface faces inwardly when the occluding sheet is in the rolled configuration. The rolled configuration of the occluding sheet has a first state and a second state, the first state of the rolled configuration having a first radius and the second state of the rolled configuration having a second radius. The second radius is greater than the first radius. The framework is biased to unroll the occluding sheet along the first dimension when the occluding sheet is in the first state or the second state of the rolled configuration. The outer catheter constrains the occluding sheet in the first state when the occlusion device is disposed in the delivery lumen, and the blood vessel constrains the occluding sheet in the second state when the occlusion device is disposed in the blood vessel. The method further involves permitting the occluding sheet to unroll from the first state to the second state in the blood vessel to cover the opening in the blood vessel wall and block blood flow from the blood vessel into the aneurysm.

Further objects, features, and advantages of the present invention will become apparent from consideration of the following description and the appended claims when taken in connection with the accompanying drawings.

### BRIEF DESCRIPTION OF THE DRAWINGS

FIG. 1 is an environmental view of a blood vessel with an aneurysm extending from the blood vessel;

FIGS. 2A-2D are perspective views of an occlusion device in an unrolled configuration in accordance with one embodiment of the present invention;

FIG. 3 is a perspective view of the occlusion device shown in FIGS. 2A-2D in a rolled configuration;

FIGS. 4A-4B are end views of the occlusion device shown in FIGS. 2A-3 in a rolled configuration;

FIG. 5 is a flowchart depicting a method of preventing blood flow from a blood vessel into an aneurysm through an opening in a blood vessel wall in accordance with another embodiment of the present invention;

FIG. 6 is an environmental break-away view of a delivery system for the occlusion device of the present invention extending to a treatment site in a patient's blood vessel;

FIG. 7 is an environmental break-away view of the occlusion device of the present invention being deployed from the delivery system in the patient's blood vessel;

FIG. 8 is an end view of the occlusion device of the present invention unrolling from a first state to a second state of the rolled configuration; and

FIG. 9 is an environmental view of the occlusion device of the present invention deployed in a blood vessel and preventing blood flow into an aneurysm through an opening in the blood vessel wall.

### DETAILED DESCRIPTION

The present invention generally provides an occlusion device for preventing blood flow from a blood vessel into an aneurysm. The invention also provides a method of preventing blood flow from a blood vessel into an aneurysm using the occlusion device of the present invention. Embodiments of the present invention enable interventionalists to prevent aneurysm expansion and rupture in patients by relieving pressure on the walls of the aneurysm.

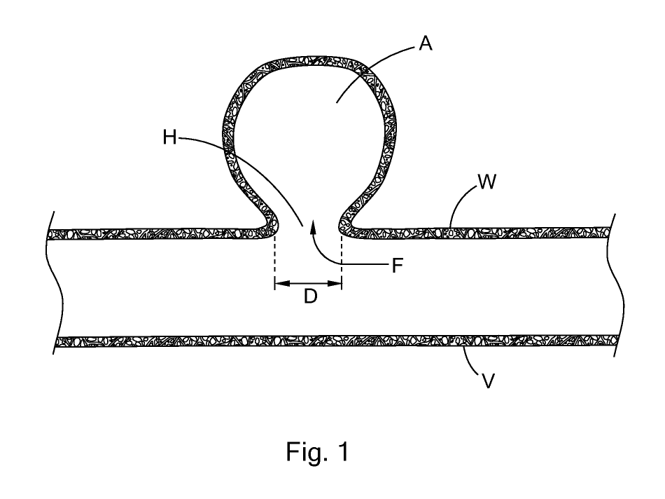

FIG. 1 shows an aneurysm A attached to a blood vessel V in a patient's vasculature. The aneurysm A is in fluid communication with the blood vessel V through an opening H in the blood vessel walls W. The opening H in the blood vessel walls W has a diameter D. Blood may flow, as indicted by the arrow F, from the blood vessel V into the aneurysm A through the opening H in the blood vessel walls W. Without treatment, the flow F of blood into the aneurysm A can cause the aneurysm to expand and possibly rupture.

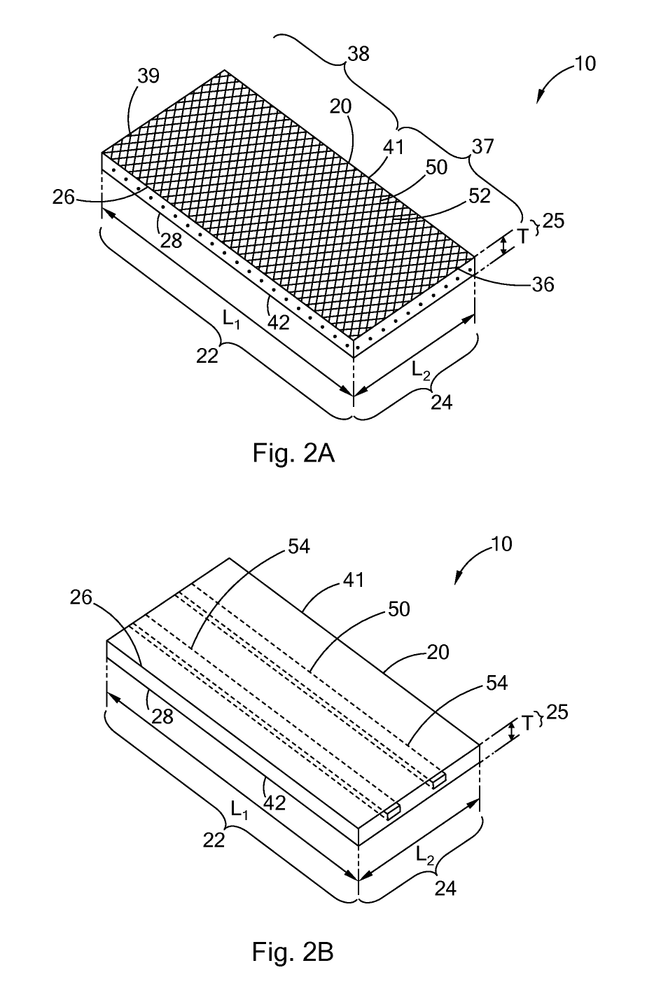

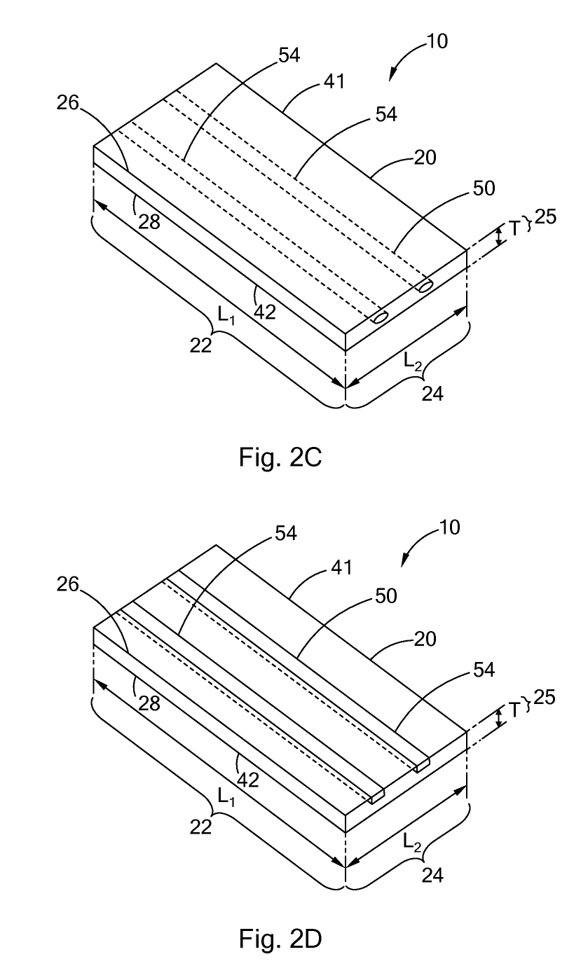

FIGS. 2A-2D illustrate several embodiments of an occlusion device 10 in accordance with the present invention. The occlusion device 10 comprises an occluding sheet 20 and a framework 50 disposed with the occluding sheet 20. The occluding sheet 20 has a first dimension 22, a second dimension 24, and a third dimension 25. The first dimension has a first length L₁. The first length L₁ will depend on the circumference of the blood vessel. In some embodiments, the first length L₁ may be greater than the circumference of the blood vessel in which the occlusion device 10 is to be deployed. For example, the first length L₁ may be 5-30% greater than the circumference of the blood vessel. The second dimension has a second length L₂. In some embodiments, the second length L₂ may be greater than the diameter D of the opening H in the blood vessel wall W leading to the aneurysm A. Preferably, the second length L₂ is between about 5 and 30 mm. The third dimension has a thickness T. Preferably, the thickness T is between about 0.06 and 0.10 mm.

The occluding sheet 20 has a first end 36, a first portion 37, a second portion 38, and a second end 39 disposed along the first dimension 22. The first portion 37 extends from the first end 36 to the second portion 38. The second portion 38 extends from the first portion 37 to the second end 39. The occluding sheet 20 also has a first edge 41 and a second edge 42 disposed at opposite ends of the second dimension 24. The occluding sheet 20 also has a first surface 26 and a second surface 28. The occluding sheet 20 may have a rectangular shape as shown in FIGS. 2A-2D. Alternatively, the occluding sheet 20 may have a square, circular, elliptical, triangular, quadrilateral, pentagonal, or other suitable shape.

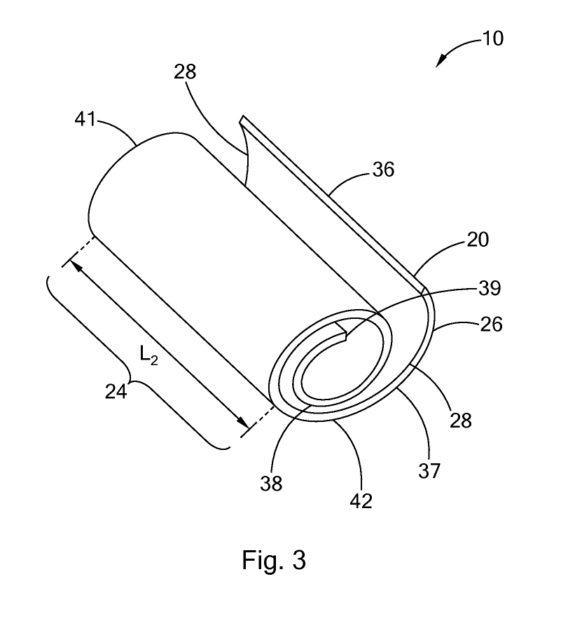

The occluding sheet 20 has an unrolled configuration (FIGS. 2A-2D) and a rolled configuration (FIG. 3). The framework 50 is omitted from FIG. 3 for the sake of clarity, but it will be understood that the framework 50 is disposed with the occluding sheet 20 when the occluding sheet 20 is in the rolled configuration. As shown in FIG. 3, the occluding sheet 20 is rolled along the first dimension to define the rolled configuration. The first portion 37 of the occluding sheet 20 may overlap the second portion 38 of the occluding sheet 20 when the occluding sheet 20 is in the rolled configuration.

The first surface 26 of the occluding sheet 20 faces outwardly when the occluding sheet 20 is in the rolled configuration. The second surface 28 of the occluding sheet 20 faces inwardly when the occluding sheet 20 is in the rolled configuration. If the first portion 37 of the occluding sheet 20 overlaps the second portion 38 of the occluding sheet 20 when the occluding sheet 20 is in the rolled configuration, the second surface 28 of the first portion 37 may be disposed against the first surface 26 of the second portion 38 when the occluding sheet 20 is in the rolled configuration. To show the details of the rolled configuration, FIG. 3 shows some space between the second surface 28 of the first portion 37 of the occluding sheet 20 and the first surface 26 of the second portion 38 occluding sheet 20. However, it will be understood that the second surface 28 of the first portion 37 of the occluding sheet 20 may contact the first surface 26 of the second portion 38 occluding sheet 20 when the occluding sheet 20 is in the rolled configuration.

When the occlusion device 10 is in the rolled configuration, the first and second edges 41 and 42 of the occluding sheet 20 define the ends of the occlusion device 10, and the second length L₂ of the second dimension 24 of the occluding sheet defines the length of the occlusion device 10. The occluding sheet 20 may be fabricated in the unrolled configuration and then rolled along the first dimension into the rolled configuration prior to deployment in a patient's blood vessel.

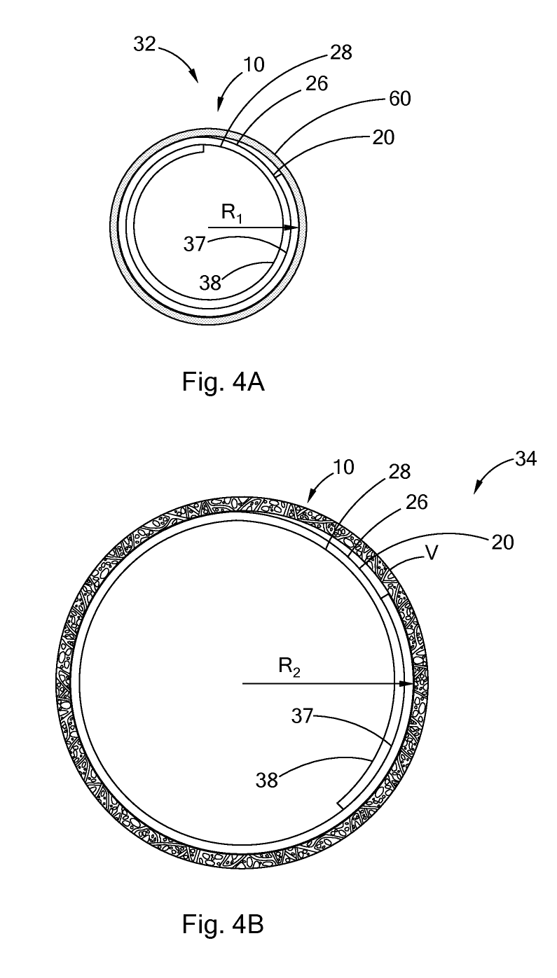

Referring now to FIGS. 4A and 4B, the rolled configuration of the occluding sheet 20 has a first state 32 and a second state 34. As shown in FIG. 4A, the first state 32 of the rolled configuration has a first radius R₁ suitable for delivery of the occlusion device 10 through a catheter 60. As shown in FIG. 4B, the second state 34 of the rolled configuration has a second radius R₂. In some embodiments, the second radius R₂ is substantially similar to the radius of the blood vessel V in which the occlusion device 10 will be deployed. Preferably, the second radius R₂ is greater than the first radius R₁. The first length of the first dimension of the occluding sheet 20 is preferably selected so that the first portion 37 of the occluding sheet 20 overlaps the second portion 38 of the occluding sheet 20 in the both the first state 32 and the second state 34 of the rolled configuration. If the second radius R₂ of the rolled configuration in the second state 34 is greater than the first radius R₁ of the rolled configuration in the first state 32, then the amount of overlap between the first portion 37 and the second portion 38 of the occluding sheet 20 will be greater in the first state 32 than in the second state 34.

The framework 50 is biased to unroll the occluding sheet 20 along the first dimension when the occluding sheet 20 is in the first state 32 or the second state 34 of the rolled configuration. In some embodiments, the framework 50 may be biased to unroll the occluding sheet 20 completely, such that the occluding sheet 20 assumes the unrolled configuration if it is not constrained in the rolled configuration. In other embodiments, the framework 50 may be biased to unroll the occluding sheet 20 partially, such that the occluding sheet 20 remains in the rolled configuration even if it is not so constrained.

The framework 50 may have any configuration suitable to unroll the occluding sheet 20 along the first dimension. For example, as shown in FIG. 2A, the framework 50 may comprise a wire mesh 52. The wires of the wire mesh 52 preferably have a cross-sectional diameter between about 0.02 mm and about 0.05 mm.

Alternatively, as shown in FIGS. 2B-2D, the framework 50 may comprise a plurality of frame members 54 disposed along the first dimension 22 of the occluding sheet 20. The frame members 54 may have a rectangular cross-section, as shown in FIGS. 2B & 2D, a round cross-section, as shown in FIG. 2C, or any other suitable cross-sectional shape. The frame members 54 preferably have a cross-sectional diameter or width between about 0.02 mm and about 0.05 mm.

In the embodiments shown in FIGS. 2B-2D, the framework 50 may include any suitable number of frame members 54. Preferably, the framework 50 includes between two and ten frame members 54. More preferably, the framework 50 includes between two and six frame members 54. Most preferably, the framework 50 includes between two and four frame members 54. Preferably, the frame members are spaced between about 1 and 3 mm apart from each other and between about 1 and 3 mm away from the first and second edges 41 and 42 of the occluding sheet 20. Thus, the number of frame members 54 disposed with the occluding sheet 20 may depend, in part, on the second length L₂ of the occluding sheet 20.

The framework 50 may be disposed on the first surface 26 of the occluding sheet 20 (FIG. 2D), on the second surface 28 of the occluding sheet 20, or between the first surface 26 and the second surface 28 of the occluding sheet 20 (FIGS. 2A-2C). Although FIG. 2D depicts frame members 54 having a rectangular cross-section, it will be understood that the wire mesh 52 or frame members 54 having a non-rectangular cross-section may also be disposed on the first surface 26 or second surface 28 of the occluding sheet 20. In some embodiments, the framework 50 may be disposed on the first surface 26 of the occluding sheet 20, i.e., on the outer surface of the occluding sheet 20 when the occluding sheet 20 is in the rolled configuration, such that the framework 50 is biased to unroll the occluding sheet 20 along the first dimension by pulling outwardly on the first and second ends 36 and 39 of the occluding sheet 20.

### Construction of Occluding Sheet

The occluding sheet 20 may be constructed from any biocompatible material suitable to provide a barrier to blood flow when deployed in a patient's blood vessel. The material should also have sufficient flexibility to be rolled into a rolled configuration as shown in FIG. 3.

As used herein, the term “biocompatible” refers to a material that is substantially non-toxic in the in vivo environment of its intended use, and that is not substantially rejected by the patient's physiological system or is non-antigenic. This can be gauged by the ability of a material to pass the biocompatibility tests set forth in International Standards Organization (ISO) Standard No. 10993; the U.S. Pharmacopeia (USP) 23; or the U.S. Food and Drug Administration (FDA) blue book memorandum No. G95-1, entitled “Use of International Standard ISO-10993, Biological Evaluation of Medical Devices Part-1: Evaluation and Testing.” Typically, these tests measure a material's toxicity, infectivity, pyrogenicity, irritation potential, reactivity, hemolytic activity, carcinogenicity, immunogenicity, and combinations thereof. A biocompatible structure or material, when introduced into a majority of patients, will not cause a significantly adverse, long-lived or escalating biological reaction or response, and is distinguished from a mild, transient inflammation which typically accompanies surgery or implantation of foreign objects into a living organism.

In some embodiments, the occluding sheet 20 may be constructed from a bioabsorbable material. As used herein, the term “bioabsorbable” refers to those materials of either synthetic or natural origin which, when placed in a living body, are degraded through either enzymatic, hydrolytic or other chemical reactions or cellular processes into by-products which are either integrated into, or expelled from, the body. It is recognized that in the literature, the terms “bioresorbable”, “bioabsorbable”, and “biodegradable” are frequently used interchangeably.

A number of bioabsorbable homopolymers, copolymers, or blends of bioabsorbable polymers are known in the medical arts. These include, but are not necessarily limited to, polyesters including poly-alpha hydroxy and poly-beta hydroxy polyesters, polycaprolactone, polyglycolic acid, polyether-esters, poly(p-dioxanone), polyoxaesters; polyphosphazenes; polyanhydrides; polycarbonates including polytrimethylene carbonate and poly(iminocarbonate); polyesteramides; polyurethanes; polyisocyantes; polyphosphazines; polyethers including polyglycols polyorthoesters; expoxy polymers including polyethylene oxide; polysaccharides including cellulose, chitin, dextran, starch, hydroxyethyl starch, polygluconate, hyaluronic acid; polyamides including polyamino acids, polyester-amides, polyglutamic acid, poly-lysine, gelatin, fibrin, fibrinogen, casein, collagen. Other bioabsorbable polymers include poly-L, D-lactide, poly-L-lactide, poly-D-lactide, bioglass, polydioxanone, polyglucanate, polylactic acid, polyelethelene oxide copolymers, tyrosine derived polycarbonate, polyglycolide, modified cellulose, poly(hydroxybutyrate), polyphosphoester, or any combination thereof.

In some other embodiments, the occluding sheet 20 may be constructed from a bioremodelable material. As used herein, the term “bioremodelable” refers to a natural or synthetic material that is bioresorbable and capable of inducing angiogenesis, tissue remodeling, or both in a subject or host. A bioremodelable material includes at least one bioactive agent capable of inducing angiogenesis or tissue remodeling. The bioactive agent(s) in the bioremodelable material may stimulate infiltration of native cells into an acellular matrix, and formation of new blood vessels (capillaries) growing into the matrix to nourish the infiltrating cells (angiogenesis). Additionally, the bioactive agent(s) may effect the degradation or replacement of the bioremodelable material by endogenous tissue. The bioremodelable material may include a naturally derived collagenous extracellular matrix (ECM) tissue structure present in, for example, native submucosal tissue sources, including, but not limited to small intestine submucosal (SIS) tissue, or it may include any one of a variety of different non-submucosal ECM-containing tissue materials or synthetic, bioresorbable non-ECM materials capable of inducing angiogenesis and tissue remodeling in a host.

The phrases “biocompatible sheet material” and “bioremodelable sheet material” refer to one or more biocompatible or bioremodelable tissue layers or synthetic polymeric layers formed into a sheet or composite thereof. A sheet of biocompatible or bioremodelable material may include, for example, extracellular matrix tissue, including one or more naturally-derived tissue layers containing an ECM scaffold, one or more biocompatible polymeric layers, or combinations thereof. The sheet of biocompatible or bioremodelable material can be in the form of a single tissue or polymeric layer or a plurality of tissue or polymeric layers in form of laminates, composites, or combinations thereof.

The terms “angiogenesis” and “angiogenic” refer to bioactive properties, which may be conferred by a bioremodelable material through the presence of growth factors and the like, which are defined by formation of capillaries or microvessels from existing vasculature in a process necessary for tissue growth, where the microvessels provide transport of oxygen and nutrients to the developing tissues and remove waste products.

The term “submucosa” refers to a natural collagen-containing tissue structure removed from a variety of sources including the alimentary, respiratory, intestinal, urinary or genital tracts of warm-blooded vertebrates. Submucosal material according to the present invention includes tunica submucosa, but may include additionally adjacent layers, such the lamina muscularis mucosa and the stratum compactum. A submucosal material may be a decellularized or acellular tissue, which means it is devoid of intact viable cells, although some cell components may remain in the tissue following purification from a natural source. Alternative embodiments (for example, fluidized compositions and the like) include submucosal material expressly derived from a purified submucosal matrix structure. Submucosal materials according to the present disclosure are distinguished from collagen materials in other closure devices that do not retain their native submucosal structures or that were not prepared from purified submucosal starting materials first removed from a natural submucosal tissue source.

The term “small intestinal submucosa” (SIS) refers to a particular submucosal tissue structure removed from a small intestine source, such as pig.

In some embodiments, the occluding sheet 20 may be constructed from a bioremodelable sheet material. When using bioremodelable sheet material as the occluding sheet 20, the bioremodelable sheet material is preferably designed to promote angiogenesis and endothelialization. In particular, the bioremodelable sheet material may capable of remodeling the surrounding tissues, such that upon implantation in a patient, the bioremodelable sheet material is degraded and replaced by the patient's endogenous tissues. As the bioremodelable sheet material is remodeled by host tissues, the aneurysm neck becomes stably closed, obviating concerns about migration of the device.

A bioremodelable sheet material may include one or more bioremodelable tissue layers formed into a sheet. The sheet material may include, for example, a single tissue layer containing ECM material, or it may include additionally adjacent tissue layers or additional tissue layers laminated together in a multilaminate structure. The sheet materials may include or be made from reconstituted or naturally-derived collagenous materials. Preferred bioremodelable materials include naturally derived tissues with ECMs possessing biotropic properties, including in certain forms angiogenic collagenous ECMs. Preferred ECMs include naturally-derived collagenous tissue materials retaining native matrix configurations and bioactive agents, such as growth factors, which serve to facilitate tissue remodeling, as opposed to collagen-based materials formed by separately purifying natural collagen and other associated components away from their native three dimensional matrix configurations or bioactive agents, including growth factors. Suitable collagenous ECMs include those derived from a variety of native tissues, including but not limited to, intestine, stomach, bladder, liver, fascia, skin, artery, vein, pericardium, pleura, heart valve, dura mater, ligament, tendon, bone, cartilage, bladder, liver, including submucosal tissues therefrom, renal capsule membrane, dermal collagen, serosa, mesenterium, peritoneum, mesothelium, various tissue membranes and basement membrane layers, including liver basement membrane, and the like. Suitable submucosa tissue materials for these purposes include, for instance, intestinal submucosa, including small intestinal submucosa, stomach submucosa, urinary bladder submucosa, and uterine submucosa. A particularly preferred ECM material is porcine SIS material. Commercially available ECM materials capable of remodeling to the qualities of its host when implanted in human soft tissues include porcine SIS material (Surgisis® and Oasis® lines of SIS materials, Cook Biotech Inc., West Lafayette, Ind.) and bovine pericardium (Peri-Strips®, Synovis Surgical Innovations, St. Paul, Minn.).

As prepared, the submucosa material and any other ECM used may optionally retain growth factors or other bioactive components native to the source tissue. For example, the submucosa or other ECM may include one or more growth factors such as basic fibroblast growth factor (FGF-2), transforming growth factor beta (TGF-beta), epidermal growth factor (EGF), and/or platelet derived growth factor (PDGF). As well, submucosa or other ECM used in the invention may include other biological materials such as heparin, heparin sulfate, hyaluronic acid, fibronectin and the like. Thus, generally speaking, the submucosa or other ECM material may include a bioactive component that induces, directly or indirectly, a cellular response such as a change in cell morphology, proliferation, growth, and/or protein or gene expression.

Submucosa or other ECM materials of the present invention can be derived from any suitable organ or other tissue source, usually sources containing connective tissues. The ECM materials processed for use in the invention will typically include abundant collagen, most commonly being constituted at least about 80% by weight collagen on a dry weight basis. Such naturally-derived ECM materials will for the most part include collagen fibers that are non-randomly oriented, for instance occurring as generally uniaxial or multi-axial but regularly oriented fibers. When processed to retain native bioactive factors, the ECM material can retain these factors interspersed as solids between, upon and/or within the collagen fibers. Particularly desirable naturally-derived ECM materials for use in the invention will include significant amounts of such interspersed, non-collagenous solids that are readily ascertainable under light microscopic examination with specific staining. Such non-collagenous solids can constitute a significant percentage of the dry weight of the ECM material in certain inventive embodiments, for example, at least about 1%, at least about 3%, and at least about 5% by weight in various embodiments of the invention.

The submucosa or other ECM material used in the present invention may also exhibit an angiogenic character and thus be effective to induce angiogenesis in a host engrafted with the material. In this regard, angiogenesis is the process through which the body makes new blood vessels to generate increased blood supply to tissues. Thus, angiogenic materials, when contacted with host tissues, promote or encourage the infiltration of new blood vessels. Methods for measuring in vivo angiogenesis in response to biomaterial implantation have recently been developed. For example, one such method uses a subcutaneous implant model to determine the angiogenic character of a material (C. Heeschen et al., Nature Medicine 7 (2001), No. 7, 833-839). When combined with a fluorescence microangiography technique, this model can provide both quantitative and qualitative measures of angiogenesis into biomaterials (C. Johnson et al., Circulation Research 94 (2004), No. 2, 262-268).

In addition to, or as an alternative to the inclusion of native bioactive components, non-native bioactive components such as those synthetically produced by recombinant technology or other methods, may be incorporated into the submucosa or other ECM tissue. These non-native bioactive components may be naturally-derived or recombinantly produced proteins that correspond to those natively occurring in the ECM tissue, but perhaps of a different species (for example, human proteins applied to collagenous ECMs from other animals, such as pigs). The non-native bioactive components may also be drug substances. Illustrative drug substances that may be incorporated into and/or onto the ECM materials used in the invention include, for example, antibiotics or thrombus-promoting substances such as blood clotting factors, for example, thrombin, fibrinogen, and the like. These substances may be applied to the ECM material as a premanufactured step, immediately prior to the procedure (for example, by soaking the material in a solution containing a suitable antibiotic such as cefazolin), or during or after engraftment of the material in the patient.

Submucosa or other ECM tissue used in the invention is preferably highly purified, for example, as described in U.S. Pat. No. 6,206,931 to Cook et al., which is incorporated by reference herein. Thus, preferred ECM material will exhibit an endotoxin level of less than about 12 endotoxin units (EU) per gram, more preferably less than about 5 EU per gram, and most preferably less than about 1 EU per gram. As additional preferences, the submucosa or other ECM material may have a bioburden of less than about 1 colony forming units (CFU) per gram, more preferably less than about 0.5 CFU per gram. Fungus levels are desirably similarly low, for example, less than about 1 CFU per gram, more preferably less than about 0.5 CFU per gram. Nucleic acid levels are preferably less than about 5 μg/mg, more preferably less than about 2 μg/mg, and virus levels are preferably less than about 50 plaque forming units (PFU) per gram, more preferably less than about 5 PFU per gram. These and additional properties of submucosa or other ECM tissue taught in U.S. Pat. No. 6,206,931 may be characteristic of the submucosa tissue used in the present invention.

A preferred purification process involves disinfecting the submucosal tissue source, followed by removal of a purified matrix including the submucosa. It is thought that delaminating the disinfected submucosal tissue from the tunica muscularis and the tunica mucosa minimizes exposure of the submucosa to bacteria and other contaminants and better preserves the aseptic state and inherent biochemical form of the submucosa, thereby potentiating its beneficial effects. Alternatively, the ECM- or submucosa may be purified a process in which the sterilization step is carried out after delamination as described in U.S. Pat. Nos. 5,993,844 and 6,572,650.

The stripping of the submucosal tissue source is preferably carried out by utilizing a disinfected or sterile casing machine, to produce submucosa, which is substantially sterile and which has been minimally processed. A suitable casing machine is the Model 3-U-400 Stridhs Universal Machine for Hog Casing, commercially available from the AB Stridhs Maskiner, Gotoborg, Sweden. As a result of this process, the measured bioburden levels may be minimal or substantially zero. Other means for delaminating the submucosa source can be employed, including, for example, delaminating by hand.

Following delamination, submucosa may be sterilized using any conventional sterilization technique including propylene oxide or ethylene oxide treatment and gas plasma sterilization. Sterilization techniques which do not adversely affect the mechanical strength, structure, and biotropic properties of the purified submucosa are preferred. Preferred sterilization techniques also include exposing the graft to ethylene oxide treatment or gas plasma sterilization. Typically, the purified submucosa is subjected to two or more sterilization processes. After the purified submucosa is sterilized, for example, by chemical treatment, the matrix structure may be wrapped in a plastic or foil wrap and sterilized again using electron beam or gamma irradiation sterilization techniques.

Bioremodelable sheet materials, including ECMs according to the present invention, may be isolated and used in the form of intact natural sheets, tissue layers, or strips, which may be optimally configured from a native, wet, fluidized, or dry formulation or states, into sheets, knitted meshes, or porous scaffolds, using one or more of the following, including stretching, chemical crosslinking, lamination under dehydrating conditions, compression under dehydrating conditions, in accordance with teachings set forth in U.S. Pat. Nos. 6,206,931 and 6,358,284; U.S. Patent Application Publication Nos. 2006/0201996, 2006/0052816, 2005/0249772, and 2004/0166169, the disclosures of which are expressly incorporated by reference herein.

In addition, bioremodelable sheet materials according to the present invention may be treated by controlled autolysis to render the materials substantially acellular and less susceptible to post-implantation mineralization as described in U.S. Pat. Nos. 5,595,571, 5,720,777, 5,843,180, 5,843,181, and U.S. Patent Application Publication Nos. 2005/020612, the disclosures of which are expressly incorporated by reference herein.

The occluding sheet 20 may also be constructed from other biocompatible sheet materials. Biocompatible sheet materials include a variety of natural or synthetic polymeric materials known to those of skill in the art which can be formed into flexible sheet materials. Exemplary biocompatible sheet materials include polymeric materials, including textile materials; fibrous materials, including thrombogenic fibrous materials; and other biocompatible sheet materials suitable for occlusion, which are known to those of skill in the art.

The biocompatible sheet materials may include porous or non-porous materials. When using non-bioremodelable synthetic sheet materials, the sheet materials are preferably made from porous materials, which can facilitate transfer of clotting factors and other bioactive agents associated with bioremodeling. A porous polymeric sheet may have a void-to-volume ratio from about 0.40 to about 0.90. Preferably the void-to-volume ratio is from about 0.65 to about 0.80. The resulting void-to-volume ratio can be substantially equal to the ratio of salt volume to the volume of the polymer plus the salt. Void-to-volume ratio is defined as the volume of the pores divided by the total volume of the polymeric layer including the volume of the pores. The void-to-volume ratio can be measured using the protocol described in AAMI (Association for the Advancement of Medical Instrumentation) VP20-1994, Cardiovascular Implants—Vascular Prosthesis section 8.2.1.2, Method for Gravimetric Determination of Porosity. The pores in the polymer can have an average pore diameter from about 1 micron to about 400 microns. Preferably the average pore diameter is from about 1 micron to about 100 microns, and more preferably is from about 1 micron to about 10 microns. The average pore diameter may be measured based on images from a scanning electron microscope (SEM).

Biocompatible sheet materials may be formed from fibers, or any suitable material (natural, synthetic, or combination thereof) that is pliable, strong, resilient, elastic, and flexible. The material should be biocompatible or capable of being rendered biocompatible by coating, chemical treatment, or the like. Thus, in general, the material may comprise a synthetic biocompatible material that may include, for example, bioresorbable materials such as polylactic acid (PLA), polyglycolic acid (PGA), polycaprolactone (PCL), polydioxanone (PDO), trimethylene carbonate (TMC), polyvinyl alcohol (PVA), and copolymers or blends thereof; polyurethanes, including THORALON® (THORATEC, Pleasanton, Calif.), as described in U.S. Pat. Nos. 4,675,361, 6,939,377, and U.S. Patent Application Publication No. 2006/0052816, the disclosures of which are incorporated by reference herein; cellulose acetate, cellulose nitrate, silicone, polyethylene terephthalate, polyamide, polyester, polyorthoester, polyanhydride, polyether sulfone, polycarbonate, polypropylene, high molecular weight polyethylene, polytetrafluoroethylene, or mixtures or copolymers thereof, a polyanhydride, polycaprolactone, polyhydroxy-butyrate valerate, polyhydroxyalkanoate, or another polymer able to be made biocompatible.

Suitable biocompatible polyurethanes, including biocompatible polyurethanes sold under the trade name THORALON® (THORATEC, Pleasanton, Calif.), are described in U.S. Pat. Nos. 4,675,361 and 6,939,377, both of which are incorporated herein by reference. Briefly, these publications describe a polyurethane base polymer (referred to as BPS-215) blended with a siloxane containing surface modifying additive (referred to as SMA-300). Base polymers containing urea linkages can also be used. The concentration of the surface modifying additive may be in the range of 0.5% to 5% by weight of the base polymer.

The SMA-300 component (THORATEC) is a polyurethane containing polydimethylsiloxane as a soft segment and the reaction product of diphenylmethane diisocyanate (MDI) and 1,4-butanediol as a hard segment. A process for synthesizing SMA-300 is described, for example, in U.S. Pat. Nos. 4,861,830 and 4,675,361, which are incorporated herein by reference.

The BPS-215 component (THORATEC) is a segmented polyetherurethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED).

THORALON® has been used in certain vascular applications and is characterized by thromboresistance, high tensile strength, low water absorption, low critical surface tension, and good flex life. THORALON® is believed to be biostable and to be useful in vivo in long term blood contacting applications requiring biostability and leak resistance. Because of its flexibility, THORALON® has been particularly useful in larger vessels, such as the abdominal aorta, where elasticity and compliance is beneficial.

THORALON® can be manipulated to provide either porous or non-porous THORALON®. Formation of porous THORALON® is described, for example, in U.S. Pat. No. 6,752,826 and 2003/0149471 A1, both of which are incorporated herein by reference. Porous THORALON® can be formed by mixing the polyetherurethane urea (BPS-215), the surface modifying additive (SMA-300) and a particulate substance in a solvent. The particulate may be any of a variety of different particulates or pore forming agents, including inorganic salts. Preferably the particulate is insoluble in the solvent. Examples of solvents include dimethyl formamide (DMF), tetrahydrofuran (THF), dimethyacetamide (DMAC), dimethyl sulfoxide (DMSO), or mixtures thereof. The composition can contain from about 5 wt % to about 40 wt % polymer, and different levels of polymer within the range can be used to fine tune the viscosity needed for a given process. The composition can contain less than about 5 wt % polymer for some spray application embodiments. The particulates can be mixed into the composition. For example, the mixing can be performed with a spinning blade mixer for about an hour under ambient pressure and in a temperature range of about 18° C. to about 27° C. The entire composition can be cast as a sheet, or coated onto an article such as a mandrel or a mold. In one example, the composition can be dried to remove the solvent, and then the dried material can be soaked in distilled water to dissolve the particulates and leave pores in the material. In another example, the composition can be coagulated in a bath of distilled water. Since the polymer is insoluble in the water, it will rapidly solidify, trapping some or all of the particulates. The particulates can then dissolve from the polymer, leaving pores in the material. It may be desirable to use warm water for the extraction, for example water at a temperature of about 60° C. The resulting void-to-volume ratio can be substantially equal to the ratio of salt volume to the volume of the polymer plus the salt. The resulting pore diameter can also be substantially equal to the diameter of the salt grains.

A variety of other biocompatible polyurethanes may be employed in the above-described materials. These include polyurethane ureas that preferably include a soft segment and a hard segment formed from a diisocyanate and diamine. For example, polyurethane ureas with soft segments such as polytetramethylene oxide (PTMO), polyethylene oxide, polypropylene oxide, polycarbonate, polyolefin, polysiloxane (i.e. polydimethylsiloxane), and other polyether soft segments made from higher homologous series of diols may be used. Segments can be combined as copolymers or as blends. Mixtures of the soft segments may also be used. The soft segments also may have either alcohol end groups or amine end groups. The molecular weight of the soft segments may vary from about 500 to about 5,000 g/mole.

The diisocyanate may be represented by the formula OCN—R—NCO, where —R— may be aliphatic, aromatic, cycloaliphatic or a mixture of aliphatic and aromatic moieties. Examples of diisocyanates include MDI, tetramethylene diisocyanate, hexamethylene diisocyanate, trimethyhexamethylene diisocyanate, tetramethylxylylene diisocyanate, 4,4′-dicyclohexylmethane diisocyanate, dimer acid diisocyanate, isophorone diisocyanate, metaxylene diisocyanate, diethylbenzene diisocyanate, decamethylene 1,10 diisocyanate, cyclohexylene 1,2-diisocyanate, 2,4-toluene diisocyanate, 2,6-toluene diisocyanate, xylene diisocyanate, m-phenylene diisocyanate, hexahydrotolylene diisocyanate (and isomers), naphthylene-1,5-diisocyanate, 1-methoxyphenyl 2,4-diisocyanate, 4,4′-biphenylene diisocyanate, 3,3′-dimethoxy-4,4′-biphenyl diisocyanate and mixtures thereof.

The diamine used as a component of the hard segment includes aliphatic amines, aromatic amines and amines containing both aliphatic and aromatic moieties. For example, diamines include ethylene diamine, propane diamines, butanediamines, hexanediamines, pentane diamines, heptane diamines, octane diamines, m-xylylene diamine, 1,4-cyclohexane diamine, 2-methypentamethylene diamine, 4,4′-methylene dianiline, and mixtures thereof. The amines may also contain oxygen and/or halogen atoms in their structures.

The hard segment may be formed from one or more polyols. Polyols may be aliphatic, aromatic, cycloaliphatic or may contain a mixture of aliphatic and aromatic moieties. For example, the polyol may be ethylene glycol, diethylene glycol, triethylene glycol, 1,4-butanediol, 1,6-hexanediol, 1,8-octanediol, propylene glycols, 2,3-butylene glycol, dipropylene glycol, dibutylene glycol, glycerol, or mixtures thereof.

Biocompatible polyurethanes modified with cationic, anionic and aliphatic side chains may also be used. See, for example, U.S. Pat. No. 5,017,664.

Other biocompatible polyurethanes include: segmented polyurethanes, such as BIOSPAN; polycarbonate urethanes, such as BIONATE; and polyetherurethanes, such as ELASTHANE; (all available from POLYMER TECHNOLOGY GROUP, Berkeley, Calif.).

Other biocompatible polyurethanes include polyurethanes having a siloxane segment, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, -20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. These polymers are synthesized through a multi-step bulk synthesis in which PDMS is incorporated into the polymer soft segment with PTMO (PURSIL) or an aliphatic hydroxy-terminated polycarbonate (CARBOSIL). The hard segment consists of the reaction product of an aromatic diisocyanate, MDI, with a low molecular weight glycol chain extender. In the case of PURSIL-AL the hard segment is synthesized from an aliphatic diisocyanate. The polymer chains are then terminated with a siloxane or other surface modifying end group. Siloxane-polyurethanes typically have a relatively low glass transition temperature, which provides for polymeric materials having increased flexibility relative to many conventional materials. In addition, the siloxane-polyurethane can exhibit high hydrolytic and oxidative stability, including improved resistance to environmental stress cracking. Examples of siloxane-polyurethanes are disclosed in U.S. Pat. Application Publication No. 2002/0187288 A1, which is incorporated herein by reference.

Biocompatible polyurethanes may be end-capped with surface active end groups, such as, for example, polydimethylsiloxane, fluoropolymers, polyolefin, polyethylene oxide, or other suitable groups. See, for example the surface active end groups disclosed in U.S. Pat. No. 5,589,563, which is incorporated herein by reference.

The polymeric materials may include a textile material. The textile includes fibers and may take many forms, including woven (including knitted) and non-woven. Preferably, the fibers of the textile comprise a synthetic polymer. Polymeric materials that can be formed into fibers suitable for making textiles include polyethylene, polypropylene, polyaramids, polyacrylonitrile, nylons and cellulose, in addition to polyesters, fluorinated polymers, and polyurethanes as listed above. Additionally preferred textiles include those formed from polyethylene terephthalate, polytetrafluoroethylene (PTFE), expanded polytetrafluoroethylene (ePTFE), PTFE, and polyesters. These materials are inexpensive, easy to handle, have good physical characteristics and are suitable for clinical application. Examples of biocompatible polyesters include DACRON (DUPONT, Wilmington, Del.) and TWILLWEAVE MICREL (VASCUTEK, Renfrewshire, Scotland).

Films or sheets of ePTFE are typically porous without the need for further processing. The structure of ePTFE can be characterized as containing nodes connected by fibrils. Porous ePTFE can be formed, for example, by blending PTFE with an organic lubricant and compressing it under relatively low pressure. Using a ram type extruder, the compressed polymer is then extruded through a die, and the lubricant is removed from the extruded polymer by drying or other extraction method. The dried material is then rapidly stretched and/or expanded at elevated temperatures. This process can provide for ePTFE having a microstructure characterized by elongated nodes interconnected by fibrils. Typically, the nodes are oriented with their elongated axis perpendicular to the direction of stretch. After stretching, the porous polymer is sintered by heating it to a temperature above its crystalline melting point while maintaining the material in its stretched condition. This can be considered as an amorphous locking process for permanently setting the microstructure in its expanded or stretched configuration. The structure and porosity of ePTFE is disclosed, for example, in U.S. Pat. Nos. 6,547,815 B2; 5,980,799; and 3,953,566; all of which are incorporated herein by reference. Structures of porous hollow fibers can be formed from PTFE, and these porous hollow fibers can be assembled to provide a cohesive porous sheet or polymeric coating. Porous hollow fibers containing PTFE are disclosed, for example, in U.S. Pat. No. 5,024,671, which is incorporated herein by reference.

Thrombogenic fibrous materials include synthetic or natural fibrous material having thrombogenic properties. Exemplary thrombogenic fibrous materials include, but are not limited to, DACRON (DUPONT, Wilmington, Del.), cotton, silk, wool, polyester thread and the like.

Textile materials may be woven (including knitted) textiles or nonwoven textiles. Nonwoven textiles are fibrous webs that are held together through bonding of the individual fibers or filaments. The bonding can be accomplished through thermal or chemical treatments or through mechanically entangling the fibers or filaments. Because nonwovens are not subjected to weaving or knitting, the fibers can be used in a crude form without being converted into a yarn structure. Woven textiles are fibrous webs that have been formed by knitting or weaving. The woven textile structure may be any kind of weave including, for example, a plain weave, a herringbone weave, a satin weave, or a basket weave.

Woven fabrics may have any desirable shape, size, form and configuration. For example, the fibers of a woven fabric may be filled or unfilled. Examples of how the basic unfilled fibers may be manufactured and purchased are indicated in U.S. Pat. No. 3,772,137, by Tolliver, disclosure of which is incorporated by reference. Fibers similar to those described are currently being manufactured by the DuPont Company from polyethylene terephthalate (often known as “DACRON” when manufactured by DuPont), and by other companies from various substances.

Preferably the textile is made of one or more polymers that do not require treatment or modification to be biocompatible. However, materials that are not inherently biocompatible may be subjected to surface modifications in order to render the materials biocompatible. Examples of surface modifications include graft polymerization of biocompatible polymers from the material surface, coating of the surface with a crosslinked biocompatible polymer, chemical modification with biocompatible functional groups, and immobilization of a compatibilizing agent such as heparin or other substances. Thus, any fibrous material may be used to form a textile material, provided the final textile is biocompatible.

Non-native bioactive agents, such as those synthetically produced by recombinant technology or other methods, may be incorporated into any of the above-described biocompatible materials. The bioactive agent may be biochemical, organic, inorganic or synthetic in nature. Preferably the bioactive agent will be thrombogenic, fibrogenic, angiogenic, antithrombolytic, antifibrinolytic, fibrin stabilizing, wound healing, fibroblast stimulatory, vascularization promoting, cell and/or tissue attachment promoting, extracellular matrix promoting and/or the like. The bioactive agent may be a protein, peptide, growth factor, peptidomimetic, organic molecule, drug, antibiotic agent, biocidal agent, synthetic molecule, synthetic polymer, or the like. Preferably, the bioactive agent will accelerate or support thrombosis, fibrosis, deposition of connective tissue (e.g., collagen etc) in or around the closure device and/or stronger anchoring of the closure device to surrounding tissues. The non-native bioactive agents may be naturally-derived or recombinantly produced proteins, such as growth factors, which are normally found in ECM tissues. These proteins may be obtained from or engineered from any animal species. The non-native bioactive agents may also be drug substances, including antibiotics and the like.

Bioactive agents that may be incorporated into or onto ECM materials used in the invention include, for example, antibiotics or thrombus-promoting substances such as blood clotting factors, for example, thrombin, fibrinogen, and the like. These substances may be applied to the biocompatible material as a premanufactured step, immediately prior to the procedure (for example, by soaking the material in a solution containing a suitable antibiotic such as cefazolin), or during or after engraftment of the material in the patient. Alternatively, the bioactive agent(s) may be incorporated into the pores of porous polymeric materials and/or they may be chemically bonded to the biocompatible material or polymer backbone using e.g., chemical cross-linking agents or other means conventionally available to those of skill in the art. By way of example, bioactive agent(s) may be embedded into the pores of the polymeric material in a range between about 0.005% w/w and 50% w/w, between about 0.05% and 10% w/w, between about 0.1% w/w and 2% w/w, between about 0.25% w/w and 1% w/w and combinations of ranges therefrom.

Exemplary bioactive agents include, but are not limited to, clotting factors, including, but not limited to plasmin, thrombin, prothrombin, fibrinogen, Factor V, Factor Va, Factor VII, Factor Vila, Factor VIII, Factor Villa, Factor IX, Factor IXa, Factor X, Factor Xa, Factor XI, Factor XIa, Factor XII, XIIa, Factor XIII, von Willebrand Factor (vWF), other coagulation cascade factors and derivatives (e.g., natural, synthetic, recombinant etc.) therefrom; antifibrinolytic agents, including, but not limited to, aminocaproic acid, aprotinin, tranexamic acid, desopressin, etamsylate; integrins; peptides containing RGD (arginine-glycine-aspartic acid) residues; cell attachment factors, including, but not limited to collagen (Types I-XIV), elastin, fibronectin, laminin, vitronectin; homocysteine; growth factors, including, but not limited to Connective Tissue Growth Factor (CTGF), Vascular Endothelial Growth Factor (VEGF), Platelet Derived Growth Factor (PDGF), Fibroblast Growth Factor (FGF), Keratinocyte Growth Factor (KGF), Tumor Necrosis Factor (TNF), Epidermal Growth Factor (EGF), Transforming Growth Factor-alpha (TGF-α), Transforming Growth Factor-beta (TGF-β); cytokines, interleukins (e.g., IL-1, -2, -6, -8 etc.), chemokines having the above described chemical or biological properties. The biocompatible material may hold a single bioactive agent or a plurality of bioactive agents, as in the form of e.g., a cocktail.

### Construction of Framework

The framework 50 of the occlusion device 10 may be constructed from any suitable material having sufficient strength and elasticity to unroll the occluding sheet 20 along the first dimension. In some embodiments, the framework 50 may be constructed from a metal or metal alloy, such as nitinol, stainless steel, magnesium, iron, or any other suitable metal or metal alloy. Preferably, the framework is constructed from a shape-memory material, and more preferably from nitinol.

In one embodiment, the framework 50 may be constructed from a nitinol alloy having a martensitic-austenitic transition temperature that is slightly below human body temperature. The framework 50 may be heat treated in a flat configuration, such that the framework 50 will be biased to unroll the occluding sheet 20 when the alloy material is in its austenitic state. In this embodiment, the occlusion device 10 may be maintained at a low temperature prior to deployment in a patient's blood vessel, such that the occluding sheet 20 remains in the rolled configuration. Upon deployment in the patient's blood vessel, the occlusion device 10 may be warmed to a temperature exceeding the transition temperature so that the framework 50 is biased to unroll the occluding sheet.

### Marker Materials

The occlusion device 10 may include radiopaque marker materials to permit imaging of the device during delivery to the aneurysm. These radiopaque materials may be used directly in the construction of certain components of the device, or they may be added to one or more components of the device so as to render those components radiopaque or MRI compatible. For example, the framework 50 of the occlusion device 10 may include radiopaque materials, fillers, marker bands, or powders. In other embodiments, the occluding sheet 20 may include marker bands along the first and second edges 41 and 42.

Exemplary radiopaque marker materials include but are not limited to, platinum, gold, tungsten, tantalum, tantalum powder, bismuth, bismuth oxychloride, barium, barium sulphate, iodine and the like. Metallic bands of stainless steel, tantalum, platinum, gold, or other suitable materials, can include a dimple pattern, which can further facilitate ultrasound or X-ray identification.

Radiopaque markers may be introduced in any form suitable for rendering the devices radiopaque or MRI compatible. In addition, the radiopaque materials can be incorporated in the devices by a variety of common methods, such as adhesive bonding, lamination between two material layers, vapor deposition, and the materials and methods described in U.S. Pat. Appl. Publ. No. 2003/0206860, the disclosure of which is incorporated herein by reference.

Method of Preventing Blood Flow from a Blood Vessel into an Aneurysm

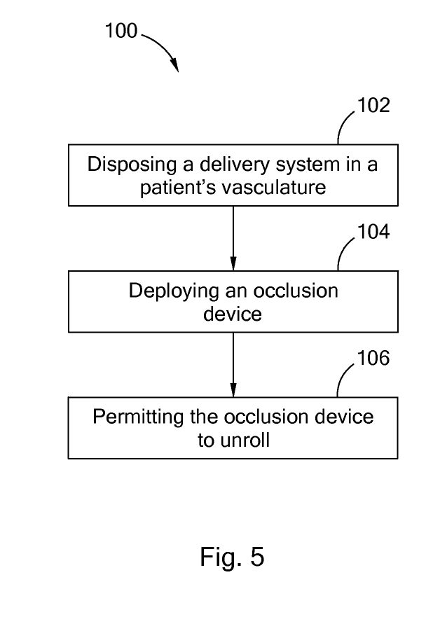

Referring now to FIG. 5, the present invention also provides a method 100 of preventing blood flow from a blood vessel into an aneurysm by deploying the occlusion device described above in the blood vessel. The method 100 may be employed to prevent blood flow into any aneurysm, including both arterial and venous aneurysms, and including but not limited to cerebral and aortic aneurysms. In one embodiment, the method 100 is employed to prevent blood flow into a cerebral aneurysm.

The size of occlusion device 10 employed in the method 100 may be selected based on the anatomical features of the blood vessel V, the aneurysm A, and the opening H, as shown in FIG. 1. For example, the second length L₂ of the occluding sheet 20 may be selected to be greater than the diameter D of the opening H in the blood vessel wall W so that the occluding sheet 20 can completely cover the opening H when the occlusion device 10 is deployed in the patient's blood vessel V. Similarly, the first length L₁ of the occluding sheet 20 may be selected to be greater than the circumference of the blood vessel V so that the first portion of the occluding sheet 20 overlaps the second portion of the occluding sheet 20 when the occlusion device is deployed in the blood vessel V.

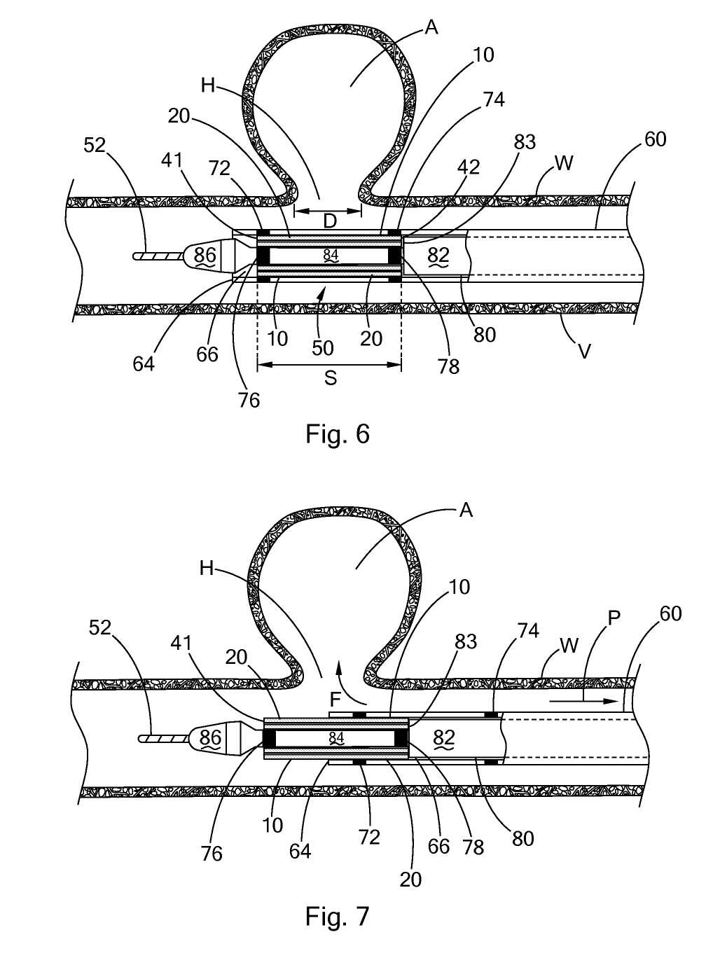

As indicated in box 102, and as illustrated in FIG. 6, the method 100 comprises disposing a delivery system 50 in a patient's vasculature. The delivery system 50 includes an outer catheter 60 having a proximal end (not shown), a distal end 64, and a delivery lumen 66 formed therethrough. The delivery system 50 may also include an inner catheter 80 means disposed in the delivery lumen 66 of the outer catheter 60 for deploying the occlusion device 10 from the outer catheter 60.

The delivery system 50 is disposed in the patient's vasculature such that the distal end 64 of the outer catheter 60 is disposed in the blood vessel V adjacent to the opening H in the blood vessel wall W, as illustrated in FIG. 6. Preferably, the distal end 64 of the outer catheter 60 is disposed in the blood vessel V distal to the opening H in the blood vessel wall W. The proximal end of the outer catheter 60 may remain outside of the patient's vasculature.

In some embodiments, the outer catheter 60 may include one or more radiopaque marker bands disposed adjacent to the distal end 64 of the outer catheter 60. As shown in FIG. 6, the marker bands may include a distal marker band 72 disposed at or near the distal end 64 of the outer catheter 60. The marker bands may further include a proximal marker band 74 disposed proximally to the distal marker band 72. The distal and proximal marker bands 72 and 74 may be separated by a distance S. The distance S may be approximately equal to the second length L₂ of the occluding sheet 20 to be used for a given application. Because the second length L₂ of the occluding sheet may be selected to be greater than the diameter D of the opening H, the distance S between the marker bands 72 and 74 may also be greater than the diameter D of the opening H. The radiopaque marker bands 72 and 74 may be formed from one or more of the radiopaque marker materials described above.

As shown in FIG. 6, the inner catheter 80 may comprise a proximal portion 82, a distal tip 86, and a recessed portion 84 extending between the proximal portion 82 and the distal tip portion 86. The inner catheter 80 may also include a shoulder 83 disposed between the proximal portion 82 and the recessed portion 84. Preferably the distal tip portion 86 is constructed from a soft, flexible material to avoid damage to the patient's vasculature.

The recessed portion 84 preferably has a length that is approximately equal to the second length L₂ of the occluding sheet 20 to be used for a given application, such that the occluding sheet 20 may be wrapped around the recessed portion 84 of the inner catheter 80 in the delivery lumen 66 of the outer catheter 60.

In some embodiments, the recessed portion 84 may include marker bands 76 and 78 disposed at its proximal and distal ends, respectively. If the length of the recessed portion is approximately equal to the second length L₂ of occluding sheet 20, the distance between the marker bands 76 and 78 will also be approximately equal to the second length L₂ of occluding sheet 20. The radiopaque marker bands 76 and 78 may be formed from one or more of the radiopaque marker materials described above.

The delivery system 50 may be disposed in the patient's vasculature by percutaneously inserting the delivery system 50 into the patient's vasculature, and then advancing the delivery system 50 through the patient's vasculature until the distal end 64 of the outer catheter 60 is disposed in the blood vessel V adjacent to the opening H in the blood vessel wall W.

In some embodiments, the step of percutaneously inserting the delivery system 50 into the patient's vasculature may involve inserting a wire guide 52 into the patient's vasculature. The wire guide 52 may be advanced through the patient's vasculature until the distal end of the wire guide 52 is disposed in the blood vessel V distal to the opening H in the blood vessel wall W. The delivery system 50 may then be inserted into the patient's vasculature over the wire guide 52. For example, the wire guide 52 may be received in the lumen of the inner catheter 80. The delivery system 50 may be inserted into the patient's vasculature at any suitable location. In some embodiments, the delivery system 50 may be inserted into the patient's vasculature through the patient's iliac artery.

In the step of advancing the delivery system 50 through the patient's vasculature, the delivery system 50 is advanced through the patient's vasculature until the distal end 64 of the outer catheter 60 is disposed in the blood vessel V adjacent to the opening H, as illustrated in FIG. 6. Preferably, the delivery system 50 is advanced through the patient's vasculature until the distal end 64 of the outer catheter 60 is disposed in the blood vessel V distal to the opening H in the blood vessel wall W. As the outer catheter 60 is advanced through the patient's vasculature, the proximal end of the outer catheter 60 may remain outside of the patient's vasculature.

In some embodiments, such as when the outer catheter 60 includes marker bands 72 and 74, the advancement of the delivery system 50 through the patient's vasculature may be monitored by imaging. As shown in FIG. 6, the distal marker band 72 may be positioned distally to the opening H in the blood vessel wall W. If the outer catheter 60 also includes a proximal marker band 74, the proximal marker band 74 may be positioned proximally to the opening H in the blood vessel wall W.

In some embodiments, the occlusion device 10 and the inner catheter 80 may already be disposed in the delivery lumen 66 of the outer catheter 60 when the outer catheter 60 is inserted into the patient's vasculature and advanced to the treatment site. More specifically, as shown in FIG. 6, the occluding sheet 20 may be wrapped around the recessed portion 84 of the inner catheter 80, with the outer catheter 60 constraining the occluding sheet 20 in the first state of the rolled configuration. As discussed above, the first portion of the occluding sheet 20 may significantly overlap the second portion of the occluding sheet 20 when the occluding sheet 20 is in the first state of the rolled configuration. Therefore, for illustrative purposes, two layers of the occluding sheet 20 are shown on each side of the recessed portion 84 in FIG. 6. However, as will be understood by one having ordinary skill in the relevant art, such significant overlap is not a requirement of the invention.

In the embodiment shown in FIG. 6, the first end 41 of the occluding sheet 20 may be aligned approximately with the marker bands 72 and 76. Similarly, the second end 42 of the occluding sheet 20 may be aligned approximately with the marker bands 74 and 78. The distal tip 86 of the inner catheter 80 may extend distally from the distal end 64 of the outer catheter 60.

Alternatively, in other embodiments, the occlusion device 10 may be inserted into the delivery lumen 66 of the outer catheter 60 through the proximal end of the outer catheter 60 after the distal end 64 of the outer catheter 60 has been advanced to the treatment site. In these embodiments, the occlusion device 10 may be inserted into the delivery lumen 66 with the occluding sheet 20 in the first state of the rolled configuration. Once the occlusion device 10 is disposed in the catheter lumen 66, the outer catheter 60 constrains the occluding sheet 20 in the first state, as shown in FIG. 6.

The occlusion device 10 may then be pushed through the delivery lumen 66 with the inner catheter 80 until the occlusion device 10 is disposed adjacent to the distal end 64 of the outer catheter 60 as shown in FIG. 6. For example, the occlusion device 10 may be disposed over the recessed portion 84 of the inner catheter 80, and the inner catheter 80 may be advanced through the delivery lumen 66 until the first edge 41 of the occluding sheet 20 is disposed distal to the opening H, and the second edge 42 of the occluding sheet 20 is disposed proximal to the opening H. If the outer catheter 60 includes marker bands 72 and 74, and the inner catheter 80 includes marker bands 76 and 78, the inner catheter 80 and the occlusion device 10 may be advanced through the delivery lumen 66 until the marker band 76 is aligned with the distal marker band 72 and the marker band 78 is aligned with the proximal marker band 74. In this manner, the occlusion device 10 may be positioned in the correct longitudinal position in the blood vessel V to block blood flow into the aneurysm A.

As indicated in box 104, and as illustrated in FIG. 7, the method 100 further comprises deploying the occlusion device 10 of the present invention from the delivery lumen 66 into the blood vessel V through the distal end 64 of the outer catheter 60. Preferably, the occlusion device 10 is deployed by retracting the outer catheter 60 in the direction indicated by the arrow P while holding the inner catheter 80 in a constant position. In this way, the inner catheter 80 may hold the occlusion device 10 in place while the catheter 60 is retracted. More specifically, the shoulder 83 of the inner catheter 80 may contact the second edge 42 of the occluding sheet 20, holding the occlusion device 10 in place.

When the distal end 64 of the outer catheter 60 reaches the second edge 42 of the occluding sheet 20, which preferably occurs when the distal end 64 of the outer catheter 60 is disposed in the blood vessel V proximal to the opening H in the blood vessel wall W, the occlusion device 10 exits from the distal end 64 of the outer catheter 60. In some embodiments, the retraction of the outer catheter 60 relative to the occlusion device 10 and the inner catheter 80 may be monitored by imaging the marker bands 72, 74, 76, and/or 78.

As indicated in box 106, the method 100 further comprises permitting the occluding sheet 20 to unroll from the first state to the second state 34 in the blood vessel V to cover the opening H in the blood vessel wall W and prevent blood flow F from the blood vessel V into the aneurysm A. As shown in FIGS. 4A and 6, the outer catheter 60 constrains the occluding sheet 20 in the first state 32 of the rolled configuration when the occlusion device 10 is disposed in the delivery lumen 66 prior to deployment. After the occlusion device 10 is deployed from the distal end 64 of the outer catheter 60, the outer catheter 60 no longer constrains the occluding sheet 20 in the first state 32. Because the framework 50 is biased to unroll the occluding sheet 20 along the first dimension, the occluding sheet 20 may partially unroll to the extent permitted by the blood vessel V.

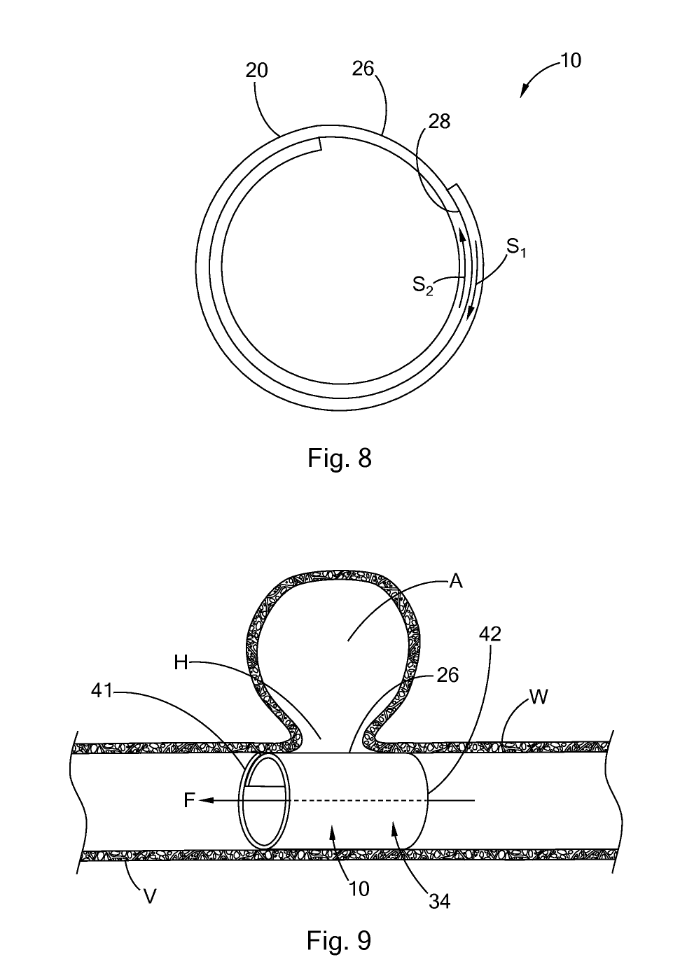

As shown in FIG. 8, the second surface 28 of the occluding sheet 20 may slide relative to first surface 26 of the occluding sheet 20 as the occluding sheet 20 unrolls from the first state 32 to the second state 34. More specifically, the first surface 26 may slide in the direction of arrow S₁, or the second surface 28 may slide in the direction of arrow S₂, or both. The occluding sheet 20 may continue to unroll until the first surface 26 of the occluding sheet 20 contacts the blood vessel wall. Thus, as shown in FIGS. 4B and 9, the blood vessel V constrains the occluding sheet 20 in the second state 34 of the rolled configuration when the occlusion device 10 is disposed in the blood vessel V after deployment. The first surface 26 of the occluding sheet 20 may contact the blood vessel wall W surrounding the opening H preventing blood flow F through the opening H into the aneurysm A. As shown in FIG. 9, the blood flow F may pass through a lumen created by the occlusion device 10.

After the occlusion device 10 exits from the distal end 64 of the outer catheter 60, and the occluding sheet 20 partially unrolls to the second state 34, the inner catheter 80 may be pulled back through the lumen created by the occlusion device 10, and both the outer catheter 60 and the inner catheter 80 can be removed from the patient's vasculature.

While the present invention has been described in terms of certain preferred embodiments, it will be understood that the invention is not limited to the disclosed embodiments, as those having skill in the art may make various modifications without departing from the scope of the following claims.

What is claimed is:

1. A method of preventing blood flow from a blood vessel into an aneurysm through an opening in a blood vessel wall, the method comprising:
disposing a delivery system in a patient's vasculature, the delivery system including an outer catheter having a proximal end, a distal end, and a delivery lumen formed therethrough, the distal end of the outer catheter being disposed in the blood vessel adjacent to the opening;
deploying an occlusion device from the delivery lumen into the blood vessel through the distal end of the outer catheter, the occlusion device comprising an occluding sheet and a framework disposed with the occluding sheet, the occluding sheet having a first dimension and a second dimension, the occluding sheet having a rolled configuration and an unrolled configuration, the occluding sheet being rolled along the first dimension to define the rolled configuration, the occluding sheet having a first surface and a second surface, the first surface facing outwardly when the occluding sheet is in the rolled configuration, the second surface facing inwardly when the occluding sheet is in the rolled configuration, the rolled configuration of the occluding sheet having a first state and a second state, the first state of the rolled configuration having a first radius and the second state of the rolled configuration having a second radius, the second radius being greater than the first radius, the framework being biased to unroll the occluding sheet along the first dimension when the occluding sheet is in the first state or the second state of the rolled configuration, the outer catheter constraining the occluding sheet in the first state when the occlusion device is disposed in the delivery lumen, the blood vessel constraining the occluding sheet in the second state when the occlusion device is disposed in the blood vessel; and
permitting the occluding sheet to unroll from the first state to the second state in the blood vessel to cover the opening in the blood vessel wall and block blood flow from the blood vessel into the aneurysm.

2. The method of claim 1, wherein the blood vessel has a blood vessel circumference and the first dimension of the occluding sheet has a first length, the first length being greater than the blood vessel circumference.

3. The method of claim 1, wherein the occluding sheet has a first end, a first portion, a second portion, and a second end disposed along the first dimension, the first portion extending from the first end to the second portion, the second portion extending from the first portion to the second end, the first portion of the occluding sheet overlapping the second portion of the occluding sheet when the occluding sheet is in the rolled configuration.

4. The method of claim 3, wherein said permitting the occluding sheet to partially unroll comprises permitting the second surface of the occluding sheet to slide relative to the first surface of the occluding sheet until the occluding sheet reaches the second state.

5. The method of claim 1, wherein the opening in the blood vessel wall has an opening diameter and the second dimension of the occluding sheet has a second length, the second length being greater than the opening diameter.

6. The method of claim 5, wherein the distal end of the outer catheter is disposed in the blood vessel distal to the opening in the blood vessel wall when the delivery system is disposed in the patient's vasculature; and
wherein said deploying an occlusion device comprises retracting the outer catheter until the distal end of the outer catheter is disposed in the blood vessel proximal to the opening in the blood vessel wall.

7. The method of claim 1, wherein the first surface of the occluding sheet contacts the blood vessel wall surrounding the opening when the occluding sheet is in the second state.

8. The method of claim 1, wherein the occluding sheet is constructed from a bioabsorbable material.

9. The method of claim 1, wherein the framework is disposed on the first surface of the occluding sheet.

10. The method of claim 1, wherein the framework is disposed on the second surface of the occluding sheet

11. The method of claim 1, wherein the framework is disposed between the first surface and the second surface of the occluding sheet.

12. The method of claim 1, wherein the framework is constructed from a shape-memory material.

13. The method of claim 12, wherein the framework is constructed from nitinol.

14. The method of claim 1, wherein the framework comprises a wire mesh.

15. The method of claim 1, wherein the framework comprises a plurality frame members disposed along the first dimension of the occluding sheet.

16. The method of claim 1, wherein the aneurysm is a cerebral aneurysm.
